(19) United States
(12) Patent Application Publication
(10) Pub. No.: US 2019/0325986 A1
(43) Pub. Date: Oct. 24, 2019

(54) METHOD AND DEVICE FOR PREDICTING AMINO ACID SUBSTITUTIONS AT SITE OF INTEREST TO GENERATE ENZYME VARIANT OPTIMIZED FOR BIOCHEMICAL REACTION

(71) Applicant: Samsung Electronics Co., Ltd., Suwon-si (KR)

(72) Inventors: Garima AGARWAL, Bangalore (IN); Tadi Venkata SIVA KUMAR, Bangalore (IN); Rajasekhara Reddy DUVVURU MUNI, Bangalore (IN); Taeyong KIM, Daejeon (KR)

(21) Appl. No.: 16/391,581

(22) Filed: Apr. 23, 2019

(30) Foreign Application Priority Data
Apr. 24, 2018 (IN) 201841015514
Oct. 2, 2018 (KR) 10-2018-0117877

(51) Int. Cl. G16B 15/30; G16B 15/20; G16B 35/20; G16B 40/20; G16B 45/00; G16C 60/00; G16C 20/50; G16C 20/64; G16C 20/70

(52) U.S. Cl. CPC G16B 15/30; G16B 15/20; G16B 35/20; G16B 40/20; G16C 20/70; G16C 60/00; G16C 20/50; G16C 20/64; G16B 45/00

(57) ABSTRACT

A method of predicting an amino acid substitution includes: receiving input of information regarding a structure of an enzyme along with the site of the enzyme in proximity to a bound ligand; identifying a functional atom of a wild type (WT) amino acid at the site of interest and a functional atom of the ligand; confirming properties of the functional atom of the WT amino acid and the functional atom of the ligand; detecting whether an interaction exists between the functional atom of the WT amino acid and the functional atom of the ligand; selecting alternative amino acids according to a result of the detecting of the interaction; determining a score for each of the selected alternative amino acids, respectively; ranking the selected alternative amino acids, based on the scores; and predicting substitutions of alternative amino acids having high rankings from among the selected alternative amino acids.

### CROSS-REFERENCE TO RELATED APPLICATIONS

This application claims the benefit of Korean Patent Application No. 10-2018-0117877, filed on Oct. 2, 2018, in the Korean Intellectual Property Office, and Indian Patent Application No. 201841015514, filed on Apr. 24, 2018, in the Indian Intellectual Property Office, the disclosures of which are incorporated herein in their entirety by reference.

### BACKGROUND

### 1. Field

The present disclosure relates to in-silico engineering (in-silico: a technology of studying vital phenomena or designing drugs or medicines using computer simulations) of an enzyme for an efficient catalysis of a given biochemical reaction. More particularly, the present disclosure relates to a method and device for predicting an amino acid substitution at a site of interest to generate an enzyme variant optimized for a biochemical reaction.

### 2. Description of the Related Art

An organism may be designed to synthesize new molecules or degrade molecules for industries related to pharmacy, energy, leather, and petroleum.

Cost-effective industrial scale production of certain materials or components requires highly efficient catalysts tuned to certain requirements and conditions.

An enzyme, which is a powerful biological catalyst, has an important role in performing a reaction in an organism. An enzyme may be inadequate or less efficient under different requirements or conditions; however, functional properties of enzymes, for example, activity, specificity, affinity, stability, or the like, may be improved through enzyme engineering. The industry seeks to improve designer enzymes for purposes such as binding to new molecules and reactions at a faster rate with higher efficiency.

Enzyme engineering and optimization are necessary steps to obtain designer enzymes having novel or improved functional properties. An enzyme may be designed to degrade/synthesize new molecules of interest, for example, a synthetic material such as plastic, or a pollutant such as tetra fluoro carbon (CF4). An enzyme may also be designed to improve existing functional properties such as activity, specificity, ligand affinity, or stability. Enzyme engineering to obtain novel/improved functional properties includes designing and screening of a large number of mutants, which is time-consuming, labor-intensive, and often includes infeasible processes.

An in-silico method may be used for rationally designing enzymes as well as limiting the number of experiments. An in-silico enzyme engineering process may include the following operations:

(i) selecting a desired enzyme scaffold,

(ii) identifying a hot spot (site) of mutants for engineering, and

(iii) introducing an alternative amino acid at a selected site to enhance functional properties of an enzyme.

While various methods may be used for operations (i) and (ii), ways to identify amino acids to be selected and lead to functional improvement of an enzyme are very limited.

Predicting a single point mutation to automatically and accurately improve a function of an enzyme is challenging for several reasons presented below:

(i) most mutants lead to function loss/reduction,

(ii) learning from data is limited due to an insufficient number of experimental data regarding a function of every site for a given ligand and changes in an amino acid, and

(iii) substitution and impacts therefrom on enzyme functions may be case-dependent due to the absence of standard rules applicable to all enzymes and sites.

Meanwhile, a paper “Binding Pocket Optimization by Computational Protein Design” (Malisi et al. Dec. 27, 2012) presents a method of designing a protein-small molecule binding, the method named POCKETOPTIMIZER. The POCKETOPTIMIZER may be used to modify protein binding pocket residues to improve or establish binding of small molecules.

The POCKETOPTIMIZER is a modular pipeline based on a number of customized molecular modeling tools to predict mutations that change the affinity of a target protein to ligands. The POCKETOPTIMIZER uses a receptor-ligand scoring function to estimate binding free energy between a protein and a ligand.

However, the POCKETOPTIMIZER may correctly predict the mutation with higher affinity only in about 69% of cases. The POCKETOPTIMIZER method, which is based on energy function-based scoring, is highly dependent on geometric compatibility, sampling changes in a side chain structure, and force fields.

### SUMMARY

Provided are a method and device for predicting an amino acid substitution at a site of interest to generate an enzyme variant optimized for a biochemical reaction.

Technical goals of the present disclosure are not limited to the above-mentioned goals, and other technical goals may be derived from embodiment to be described hereinafter.

Additional aspects will be set forth in part in the description which follows and, in part, will be apparent from the description, or may be learned by practice of the presented embodiments.

According to an aspect of an embodiment, an in-silico method of predicting an amino acid substitution at a site of interest to generate an enzyme variant optimized for a biochemical reaction includes: receiving input information regarding a structure of an enzyme along with the site of the enzyme in proximity to a bound ligand; identifying a functional atom of a wild type (WT) amino acid at the site of interest and a functional atom of the ligand; confirming properties of the functional atom of the WT amino acid and the functional atom of the ligand; detecting whether a presence or an absence of an interaction exists between the functional atom of the WT amino acid and the functional atom of the ligand; selecting alternative amino acids according to a result of the detecting of the interaction; determining scores for the selected alternative amino acids, respectively; ranking the selected alternative amino acids, based on the scores; and predicting, for optimizing an enzyme, substitutions of alternative amino acids having high rankings from among the selected alternative amino acids.

According to another aspect of an embodiment, a device for in-silico predicting an amino acid substitution at a site of interest to generate an enzyme variant optimized for a biochemical reaction includes: a memory; and a processor connected to the memory, wherein the processor performs: receiving input of information regarding the site of interest of an enzyme in proximity to a bound ligand and a structure of the enzyme; identifying a functional atom of a wild type (WT amino acid at the site of interest and the functional atom of the ligand; confirming properties of the functional atom of the WT amino acids and the functional atom of the ligand; detecting whether a presence or an absence of an interaction exists between the functional atom of the WT amino acid and the functional atom of the ligand; selecting alternative amino acids according to a result of the detecting of the interaction; determining a score for the selected alternative amino acids, respectively; ranking the alternative amino acids, based on the scores; and predicting, for optimizing the enzyme, substitutions of alternative amino acids having high rankings from among the selected alternative amino acids.

### BRIEF DESCRIPTION OF THE DRAWINGS

These and/or other aspects will become apparent and more readily appreciated from the following description of the embodiments, taken in conjunction with the accompanying drawings in which:

FIG. 1 illustrates context characterization according to an embodiment;

FIG. 2A illustrates a method adopted for selecting amino acid when an interaction is detected, according to an embodiment;

FIG. 2B illustrate a method adopted for selecting an amino acid when an interaction is not detected, according to an embodiment;

FIG. 3 is a flowchart presenting a method of predicting an amino acid substitution at a site of interest to generate an enzyme variant optimized for a biochemical reaction, according to an embodiment;

FIG. 4 is a block diagram of a device for in-silico predicting an amino acid substitution at a site of interest to generate an enzyme variant optimized for a biochemical reaction, according to an embodiment;

FIG. 5 is a diagram of a method adopted for optimizing binding of a ligand at the same site of interest in an enzyme, according to an embodiment; and

FIG. 6 is a diagram of a method adopted for optimizing binding of two ligands for the same sight of interest in a given enzyme, according to an embodiment of the present disclosure.

### DETAILED DESCRIPTION

Terms used in the present disclosure are selected from among common terms that are currently widely used in consideration of their function in the present disclosure. However, the terms may be different according to an intention of one of ordinary skill in the art, a precedent, or the advent of new technology. In some cases, some terms may be arbitrarily chosen; in those cases, meanings of the terms will be described in detail in the description of related embodiments. Accordingly, the terms used in the present disclosure will be defined based on the meaning of the terms and the entire content of the description of the present disclosure.

In descriptions regarding embodiments, it will be understood that when an element is referred to as being “connected” to another element, it may be “directly connected” to the other element or “electrically connected” to the other element with intervening elements therebetween. In the present disclosure, it will be understood that the term such as “including” or “having” are not intended to preclude the possibility in the existence of other elements and intended to indicate that one or more other elements may be added. In addition, the terms such as “unit” or “module” will be understood as a unit that processes at least one function or operation and that may be embodied in a hardware manner, a software manner, or a combination of the hardware manner and the software manner.

The terms such as “comprise” or “includes” used in the specification will not be construed as necessarily including all of elements or operations written in the specification; some elements or operations may not be included, or alternatively, one or more elements or operations may be additionally included.

Descriptions regarding the following embodiments will not be construed as limiting the scope of the present disclosure, and descriptions that may be easily derived by one of ordinary skill in the art will be construed as being included in the scope of the present disclosure. Hereinafter, embodiments merely for examples will be described in detail with reference to the attached drawings. As used herein, the term “and/or” includes any and all combinations of one or more of the associated listed items. Expressions such as “at least one of,” when preceding a list of elements, modify the entire list of elements and do not modify the individual elements of the list.

The present embodiments provide a method and device for substituting an amino acid at a site of interest to generate an enzyme variant optimized for a biochemical reaction.

The present embodiments provide a computer executing an in-silico method for predicting an amino acid substitution at a site of interest to generate an enzyme variant optimized for a biochemical reaction.

In the present embodiments, an amino acid directly interacting with a ligand is used as a target, and ligand binding may be improved using suggested mutations. More particularly, in the present embodiments, when an enzyme, a bound ligand complex, and a site of interest are given, amino acids to be substituted to improve enzyme functions may be ranked.

The present embodiments provide a rapid and accurate method of predicting which amino acid is to work at a site of interest.

By using the in-silico method, the number of experiments required for enzyme optimization may be limited, and thus, resources may be efficiently used and cost for the experiments may be reduced.

Accordingly, the present disclosure is closely related to application of industry of large-scale synthesis and decomposition of molecules of interest using microbes.

The method provided in the present embodiment may include four operations presented below:

1. characterizing a context

2. selecting an amino acid

3. ranking an amino acid

4. predicting an amino acid

Once input of information regarding a structure of a given enzyme along with a site of the enzyme to be optimized in proximity to a bound ligand is received, functional requirements to be met at the site of interest may be realized.

The context characterization may include operations as below:

(a) identifying functional atoms respectively from a wild type (WT) amino acid in the site of interest of the enzyme and from the bound ligand; and

(b) confirming properties of the identified functional atoms from the WT amino acid and the bound ligands.

Through the above-mentioned operations, the functional atoms may be identified from the ligand and the enzyme (the enzyme to be optimized), and properties of the ligand and the enzyme to be optimized may be defined according to the following aspects:

(i) properties of atoms (polar/non-polar/aromatic)

(ii) interactions between the functional atom of the enzyme and the functional atom of the ligand (aromatic/polar/hydrophobic/electrostatic)

(iii) a distance between the functional atom of the WT amino acid and the functional atom of the ligand; and

(iv) a distance between a Cα atom of the WT amino acid and the functional atom of the ligand.

Calculations and assessments according to the above-mentioned aspects may characterize the context (e.g., a microenvironment of the functional site of the enzyme). Feasibility of the amino acid substitution depends on the microenvironment of the functional site of the enzyme.

Accordingly, the embodiments may include automatic characterization of the context of the site of the enzyme and selection of the amino acid, and the amino acid may be selected based on physico-chemical properties that fit the context and priorities based on various properties.

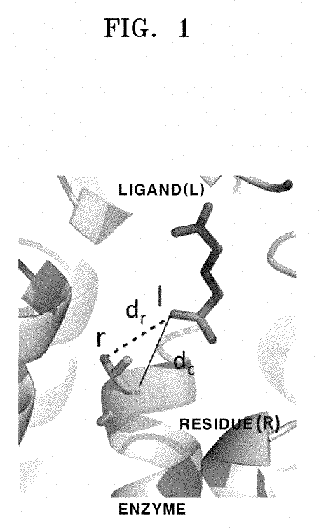

FIG. 1 illustrates context characterization according to an embodiment.

An enzyme, an amino acid residue R, and a bound ligand L are illustrated. ‘r’ is a functional atom of a WT residue R in the enzyme, and ‘I’ is a functional atom in the bound ligand L of interest. ‘dᵣ’ is a distance from the functional atom I of the bound ligand L to the functional atom r of the WT residue R, and ‘d_{c}’ is a distance from the functional atom I of the bound ligand L to a Cα atom of the WT residue R.

Definitions and properties of the functional atoms are presented as below:

{r}□R and {l}□L, Properties: dᵣ,d_{c},{I}=g(a,p,h) [Equation 1]

In [Equation 1], r, I, dᵣ, d_{c}, a, p, and h respectively indicate:

r: the functional atom(s) of WT residue (R)

I: the interacting atom(s) of ligand (L)

d_{c}: the distance between the Cα atom of R and I

dᵣ: the distance between the functional atom r and the functional atom I

a: aromatic

p: polar

h: hydrophobic

In addition, properties of the interactions between the ligand and the enzyme are presented as below:

Int{r,l}=f(a,p,h,c) [Equation 2]

In [Equation 2], Int, a, p, h, and c respectively indicate:

Int: an interaction between r and I

a: an aromatic interaction (π-π, cation-π, S-π)

p: a polar interaction (hydrogen bond)

h: a hydrophobic interaction

c: an electrostatic interaction

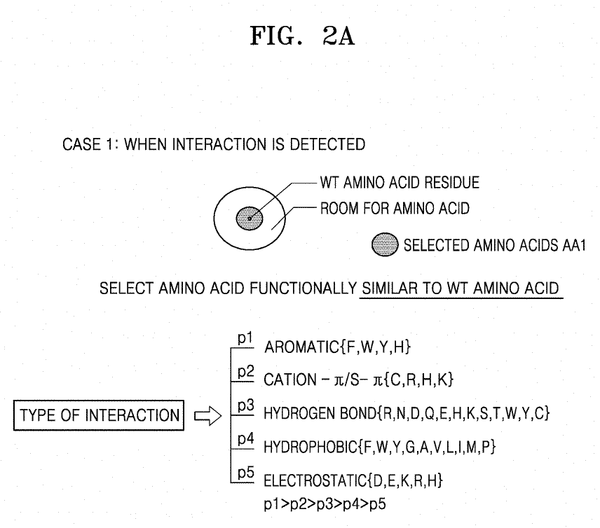

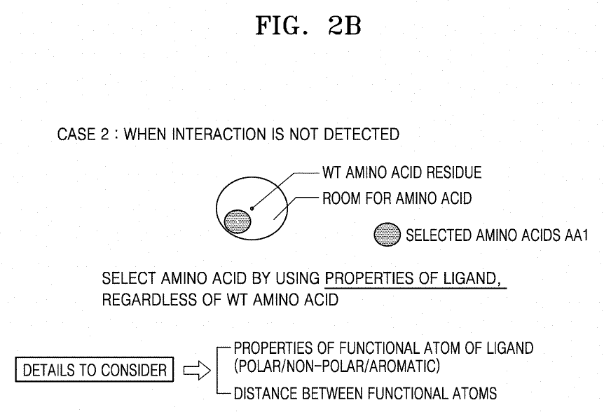

FIG. 2A is a diagram of an approach or method adopted for selecting amino acids when presence of an interaction is detected, according to an embodiment.

In an embodiment, twenty (20) standard amino acids may be grouped in various ways on the basis of their physico-chemical properties.

Table 1 presented below is an example of a method of grouping amino acids.

TABLE 1

| Physico-Chemical | |
|---|---|
| Properties | Amino Acids |
| Hydrophobic | A C T H K W Y F M L V I |
| Aromatic | F Y W H |
| Containing sulfur | M C |
| Aliphatic | L V I |
| Hydroxylic | T S |
| Charged | H K R D E |
| Polar | Y W H K R D E N Q C T S |
| Basic | H K R |
| Acidic | N Q |
| Small | V A C T P G S D N |
| Tiny | A C S G |

Two different approaches or methods are adopted according to the presence or absence of the interactions detected between the WT amino acid and the ligand.

Referring to FIG. 2A, when the interactions are detected between the ligand and the WT amino acid, selection of the amino acids may depend on the extent of functional similarity between the ligand and the WT amino acid. Amino acids causing similar interactions may be selected based on types of the detected interactions. Furthermore, the amino acids selected based on the types of interactions may be given a preset or user-defined preference order, for example, p1>p2>p3>p4>p5.

Referring to FIG. 2B, when interactions are not detected between the ligand and the WT amino acid, the amino acids may be selected based on properties of ligand atoms and the distances d_{c} and dᵣ between the functional atoms, regardless of the WT amino acid. The selected amino acids may include a set of amino acids that matches the defined constraints and functional requirements.

A result of selecting the amino acid may be a group of one or more amino acids such as AA1={A1, A2, . . . An} (n<19) that matches constraints defined according to the context.

In following operations, each of the selected amino acids may be scored for estimating ranks. The ranks are required for prioritizing amino acids which may be experimentally tested for optimizing the enzyme.

Scores may be determined by calculating properties of the amino acids, for example, volume, polarity index, and total number of hydrogen bonds formed by given amino acids. The scores may also be determined using an evolutionary property that lists the differences in substituting one amino acid with a different amino acid in a set of related enzymes and/or a given context.

$$Score \{ {AA}_{2} \} = \frac{\begin{matrix}{w}_{1} Δ v + {w}_{2} Δ pol + {w}_{3} Δ hbond + \\ {w}_{4} Δ ss + {w}_{5} sp + {w}_{6} sp 2 + {w}_{7} h\end{matrix}}{{w}_{1} + {w}_{2} + {w}_{3} + {w}_{4} + {w}_{5} + {w}_{6} + {w}_{7}} \qquad [ Equation 3 ]$$

In Equation 3, Δv, Δpol, Δhbond, Δss, sp, sp2, and h respectively indicate:

Δv: change in volume with respect to the WT amino acid

Δpol: change in polarity with respect to the WT amino acid

Δh bond: change in the total number of hydrogen bonds with respect to the WT amino acid

Δss: change in a secondary structure propensity with respect to the WT amino acid

sp: feasibility of substitution of homologues

sp2: substitution probability environment specific matrix

h: frequency of occurrence of the residue in homologues at the sites (evolutionary information)

The scores may be determined according to a distance-based measurement that calculates differences in various properties between the WT amino acid and each of the amino acid AA1 in the selected amino acid set.

In selecting amino acids having profitable properties, the goal is to minimize differences between properties of the amino acids and properties of the WT amino acid.

The scores may be calculated based on Equation 3 presented above. The amino acids are ranked based on the scores, and the lower the score, the higher the rank.

To optimize the enzyme, amino acid substitution to the selected highly ranked alternative amino acids may be predicted at one or more sites of interest.

Therefore, top-ranked amino acids may be selected for experiments.

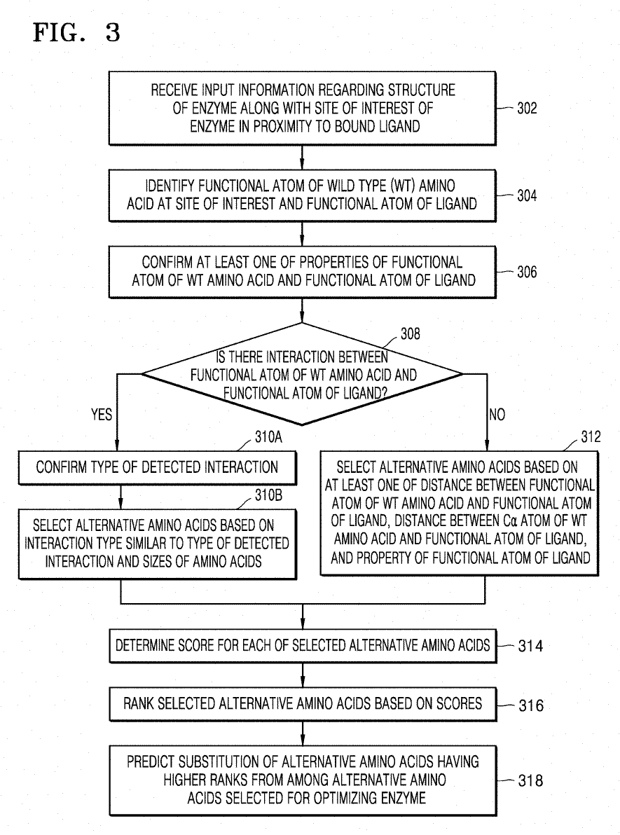

FIG. 3 is a flowchart of a method of predicting amino acid substitutions at the sites of interest for generating an enzyme variant optimized for a biochemical reaction according to an embodiment.

In operation 302, input of information regarding a site of interest of an enzyme in proximity to the bound ligand, WT amino acid R, and a structure of the enzyme may be received.

In operation 304, the functional atom of the WT amino acid in the site of interest and the functional atom of the ligand may be identified. Identifying the functional atoms is a first step of the context characterization.

The identifying of the functional atom from the WT amino acid at the site of interest in the enzyme may include identifying (a) a polar atom of a side chain of the amino acid, (b) a non-polar atom of the side chain of the amino acid, (c) a centroid of a polar atom of a polar amino acid, (d) a centroid of a non-polar atom of a non-polar amino acid, or (e) a user-defined atom, or a combination of the atoms based on a result of assessment on input information regarding the knowledge library.

The knowledge library (e.g., one or more databases) may, but is not limited to, include the data regarding the structure of the amino acids, a list of the functional atoms, a binding pocket of the enzyme, the amino acids and interaction types, the amino acids and preferred secondary structures, the amino acids and the number of hydrogen bonds, various physico-chemical properties, feasibility of substitutions in homologues, and an environment-specific substitution matrix.

The binding pocket of the enzyme, which may also be defined by the user, may provide greater flexibility to the user.

Identifying the functional atom from the bound ligand may include the following operations:

(a) calculating a distance between the functional atom in the WT amino acid and the atoms of the bound ligand; and

(b) selecting, as a following operation, a functional atom from the bound ligand, based on the calculated distance.

In operation (b), atoms mentioned below may be selected as the functional atom of the bound ligand:

(i) an atom of the bound ligand in a greatest proximity to the functional atom of the WT amino acid;

(ii) an atom of the bound ligand present within a predefined distance from the functional atom of the WT amino acid; or

(iii) an atom of the bound ligand, the atom generated by a combination of (i) and (ii).

The functional atoms identified from the WT amino acid and the ligand may be polar, non-polar, or aromatic.

In operation 306, which is the second and last operation in the context characterization, at least one property of the functional atom of the WT amino acid and the functional atoms of the ligands may be confirmed according to the following aspects:

(a) natures of all of the identified functional atoms;

(b) a distance between the functional atom of the WT amino acid and the functional atom of the ligand; and

(c) a distance between the Cα atom of the WT amino acid and the functional atom of the ligand.

In operation 308, selecting of the amino acid may begin, and the presence or absence of an interaction between the functional atom of the WT amino acid at a certain site of the enzyme and the ligand may be confirmed.

Based on the presence or absence of the interaction, the embodiments may provide two different routes.

When the presence of the interaction is detected in operation 308, operation 310A may be performed. Operation 310A may include identifying a type of the detected interaction.

The type of interaction between the functional atom of the WT amino acid at the certain site of the enzyme and the functional atom of the ligand may include an aromatic interaction, a cation-r interaction, an S-r interaction, a hydrogen bond, a hydrophobic interaction, an electrostatic interaction, or a set of user-defined interactions.

In operation 310B, alternative amino acids may be selected based on sequential sub-operations as below:

(A) selecting, from the knowledge library, amino acids having similar types of interactions; and

(B) re-selecting the selected amino acids, based on a distance between the functional atom of the WT amino acid and the functional atom of the ligand and based on sizes of the amino acids.

Operation (A) may be performed by selecting a set of amino acids based on a preference order assigned according to the types of interactions and further selecting alternative amino acids from among the selected set of amino acids based on a distance.

The preference order assigned according to the type of interaction could be a preset order such as the aromatic>cation-π/S-π>hydrogen bond>hydrophobic>and electrostatic, or according to a user-defined order.

The selection of the amino acids from the one or more selected amino acid sets, based on distances, may be performed according to the following standards:

(a) when the distance between the functional atom of the WT amino acid and the functional atom of the ligand is within a predefined cutoff size range, similar-sized amino acids are selected from the knowledge library;

(b) when the distance between the functional atom of the WT amino acid and the functional atom of the ligand is less than the predefined cutoff size range, smaller-sized amino acids are selected from the knowledge library; and

(c) when the distance between the functional atom of the WT amino acid and the functional atom of the ligand is greater than the predefined cutoff size range, larger-sized amino acids are selected from the knowledge library.

When an interaction is not detected in operation 308, operation 312 may be performed.

Operation 312 may include selecting alternative amino acids having suitable properties for the sites of the enzyme based on the following standards:

(A) nature of the functional atom of the ligand; and

(B) a distance between the functional atom of the WT amino acid and the functional atom of the ligand; or a distance between the Cα atom of the WT amino acid and the functional atom of the ligand.

In operation (A), the nature of the identified functional atom of the ligand may be computed to confirm whether the functional atom is polar, non-polar, or aromatic.

Once the nature of the identified functional atom of the ligand is confirmed, alternative amino acids having natures similar to the nature of the functional atom of the ligand may be selected from the knowledge library.

The following descriptions explain circumstances in which the above-mentioned distances in operation (B) are used for selecting the alternative amino acids.

Case 1: when the distance between the Cα atom of the WT amino acid and the functional atom of the ligand is greater than the distance between the functional atom of the WT amino acid and the functional of the ligand, the distance between the functional atom of the WT amino acid and the functional atom of the ligand may be used for selecting alternative amino acids, from the set of alternative amino acids, having natures similar to the nature of the functional atom of the ligand. By doing so, amino acids having suitable sizes may be selected by confirming an orientation of a side chain of the WT amino acid.

Case 2: when the distance between the Cα atom of the WT amino acid and the functional atom of the ligand is less than or equal to the distance between the functional atom of the WT amino acid and the functional atom of the ligand or when an enzyme structure provided in the input is of poor quality, the distance between the Cα atom of the WT amino acid and the functional atom of the ligand may be used for further selecting, from the set of the alternative amino acids, alternative amino acids having natures similar to the nature of the functional atom of the ligand. By doing so, an orientation of the side chain of the WT amino acid (which is apart from the ligand) toward the ligand is confirmed and amino acids having suitable sizes may be selected.

Case 3: the selection of the alternative amino acid from the set of alternative amino acids may be performed as a user selects one of the distance between the functional atom of the WT amino acid and the functional atom of the ligand and the distance between the Cα atom of the WT amino acid and the functional atom of the ligand.

In operation 314, scores for the selected alternative amino acids may be determined according to Equation 3.

The scores may include calculating a weighted average of volume, polarity index, a total number of hydrogen bonds formed by the given amino acid, secondary structure propensity, feasibility of substitution in homologues, frequency of substitution in user-defined homologues, and physico-chemical properties of user-defined amino acids, and the like.

In operation 316, the selected alternative amino acids may be ranked based on the scores determined in operation 314. Higher ranks may be given to the amino acids having lower scores.

In operation 318, substitutions of alternative acid substitution from among the alternative amino acids selected for optimization of the enzyme may be predicted.

In another embodiment of the present disclosure, when an enzyme has multiple sites of interest, amino acid substitution may be predicted for each of the sites in a binding pocket of the enzyme.

An enzyme variant may be optimized for the biochemical reaction, based on the predicted amino acid substitution. Information regarding the binding pocket of the enzyme may either be brought from the knowledge library, or alternatively, a user may define a binding pocket for a given enzyme in an input.

In an additional embodiment of the present disclosure, multiple bound ligands may be present in the input for the same site of interest, and the ability of each bound ligand to bind to the binding pocket of the same enzyme may be ranked by performing the operations of:

(a) assessing compatibility of each of the ligands at WT sites of the binding pocket; and

(b) prioritizing the ligands for binding, based on the assessed compatibility.

In an embodiment, the prioritization of the ligands may be performed based on the number of compatible sites.

The compatibility of binding to each ligand at the WT site is assessed based on the presence or absence of interactions with the functional atom of the ligand. The sites at which the interactions are detected may be considered suitable. It is understood that different ligands for a given enzyme may be analyzed one at a time.

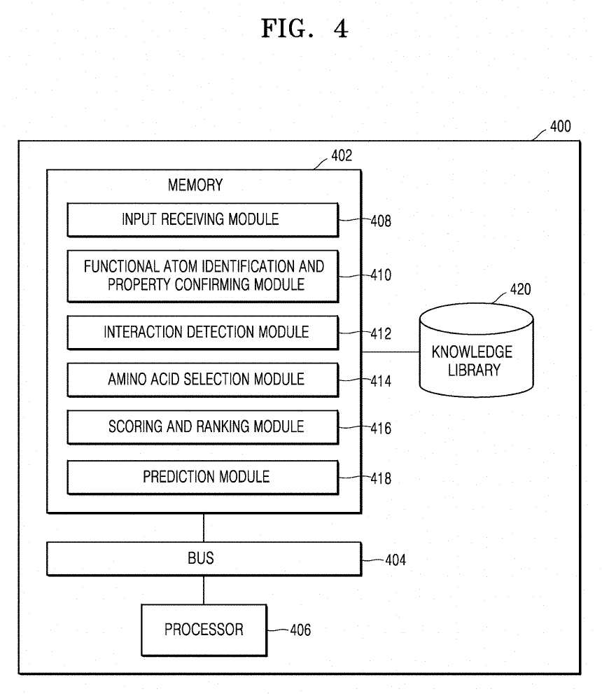

FIG. 4 is a block diagram of a device 400 for in-silico prediction of an amino acid substitution at a site of interest to generate an enzyme variant optimized for a biochemical reaction.

The device 400 may be configured to predict an amino acid substitution at a site of interest in a given enzyme. The device 400 may include a processor 406, a memory 402 coupled to the processor 406, and a database, or knowledge library, 420.

The knowledge library 420 is stored in the device 400. In another embodiment, the knowledge library 420 may be stored at a communicatively connected server (not shown in FIG. 4).

The processor 406 may be implemented as any type of computational circuit, for example, a microprocessor, a microcontroller, a complex instruction set computing (CISC) microprocessor, a reduced instruction set computing (RISC) microprocessor, a very long instruction word (VLIW) microprocessor, an explicitly parallel instruction computing (EPIC) microprocessor, a digital signal processor (DSP), another type of processing circuit, or a combination thereof.

The memory 402 includes a plurality of modules stored in the form of an executable program, or instructions, which instructs the processor 406 to perform the operations illustrated in FIG. 3.

The memory 402 may include an input receiving module 408, a functional atom identification and property confirming module 410, an interaction detection module 412, an amino acid selection module 414, a scoring and ranking module 416, and a prediction module 418.

Computer memory elements may include any suitable memory devices for storing data and an executable program, such as read only memory (ROM), random access memory (RAM), erasable programmable read only memory (EPROM), electrically erasable programmable read only memory (EEPROM), hard drive, removable media drive for memory cards, etc. Embodiments may be implemented in conjunction with program modules; the embodiments may be implemented to include functions, procedures, data structures, and application programs; the embodiments may also be implemented to perform tasks; or the embodiments may be implemented to define abstract data types (ADT) or low-level hardware contexts. Executable programs stored in any of the above-mentioned storage media may be executed by the processor 406.

The input receiving module 408 may instruct the processor 406 to perform operation 302.

The functional atom identification and property confirming module 410 may instruct the processor 406 to perform operations 304 and 306.

The interaction detection module 412 may instruct the processor 406 to perform operation 308.

The amino acid selection module 414 may instruct the processor 406 to perform operations 310A, 310B, or 312.

The scoring and ranking module 416 may instruct the processor 406 to perform operations 314 and 316.

The prediction module 418 may instruct the processor 406 to perform operation 318.

In another embodiment of the device 400, the prediction module 418 may further be configured to instruct the processor 406 to perform the following operations:

(a) predicting amino acid substitution for each of the sites in binding pocket of the enzyme; and

(b) generating an enzyme variant optimized for a biochemical reaction, based on the predicted amino acid substitution.

In another embodiment of the device 400, the scoring and ranking module 416 may further be configured to instruct the processor 406 to rank the ability of the ligands to be bound with the binding pocket of the same enzyme by performing the operations of:

(a) assessing compatibility of ligands in the WT sites of the binding pocket; and

(b) prioritizing the ligands for binding, based on the assessed compatibility.

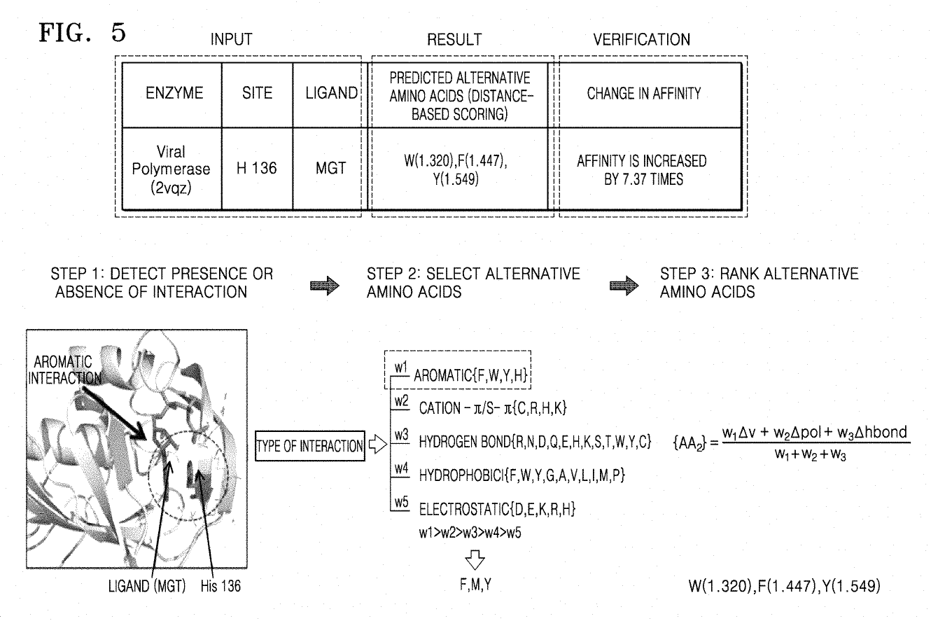

FIG. 5 is a diagram of an approach or method for binding optimization of a ligand in the same site of interest in the enzyme.

The site of interest in the enzyme may be optimized for improving bonding to the ligand.

The operations included in the embodiment are performed for the input information regarding enzyme viral polymerase 2vqz, a site of interest H136, and a bound ligand MGT, wherein WT protein has the site of interest H136 in a ligand bonding region of the bound ligand MGT.

A functional atom at the site of interest H136 was identified to form an aromatic interaction with the functional atom of the bound ligand MGT. Based on the detected interaction types, alternative amino acids W, F, and Y causing aromatic interactions were selected and ranked for substitution.

W scored the lowest (1.320) and thus ranked the highest, followed by F (1.447) and Y (1.549). Therefore, substitution of W may be predicted.

Referring to FIG. 5, substituting H to W may increase the affinity of the enzyme with the ligand by at least seven (7) times.

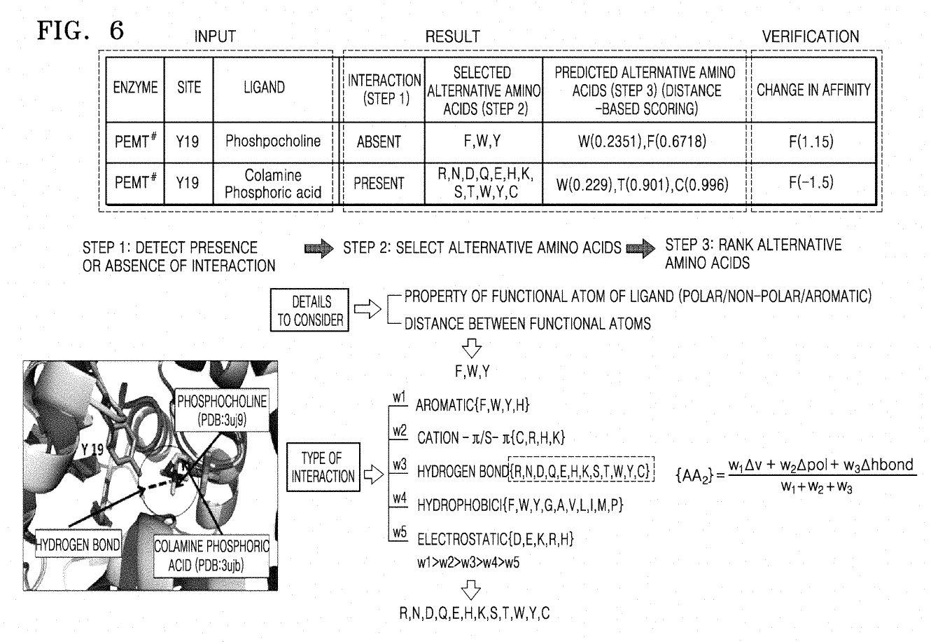

FIG. 6 is a diagram of an approach or method for binding optimization of two ligands in the same site of interest in the enzyme.

Referring to FIG. 6, it is understood that different substitutions are required in order to optimize binding to different ligands.

The input information that is received is an enzyme PEMT, a site Y19 to be bound to two different ligands (phosphocholine and colamine phosphoric acid).

For phosphocholine, no interaction was detected, and amino acids F, W, Y were selected. However, a hydrogen bond interaction was identified for the colamine phosphoric acid, and amino acids selected were R, N, D, Q, E, H, S, T, W, Y, C.

Furthermore, scores for the amino acids selected for phosphocholine were determined; the scores for W and F were 0.2351 and 0.6718, respectively.

Likewise, each of the amino acids selected with respect to the colamine phosphoric acid was scored, and the scores of W, T, C were respectively determined as 0.229, 0.901, and 0.996.

For the enzyme PEMT, in the case of phosphocoline, it is understood that the affinity increased due to substitution to F.

However, in the case of colamine phosphoric acid, F is not a suitable substituent and thus is not selected, for mutation to F has unfavorable influences on proposed functions.

The in-silico method and device in the present disclosure may be used to predict alternative amino acids at selected sites to improve functional properties of the enzyme that efficiently catalyzes a given biochemical reaction.

The embodiments may effectively exclude the mutations leading to loss/reduction of functions and predict substitutions of certain amino acids to obtain target customized enzymes.

Each site in the binding pocket of the enzymes may be systematically modified to enhance bonding to a ligand of interest.

The method may be used to prioritize ligands by optimizing bindings to different ligands or assessing bindings to different ligands.

The method may have higher accuracy compared to existing technology. In addition, the method is independent of force fields and may not be sensitive to selection of force fields.

The scores are not influenced by side chain conformers of the WT amino acid and thus may avoid sampling bias. The method may also be adopted when a structure of a ligand-enzyme complex is available for a portion of the ligand. Distance-based scoring may minimize the deviation from the WT amino acid residue.

A device according to the embodiments may include a memory to store and execute program data, a permanent storage such as a disk drive, a user interface like a communication port to communicate with external devices, a touch panel, a key, a button, and the like. The methods implemented as a software module or algorithm may be stored in a computer-readable recording medium, as computer-readable code or computer-readable program instructions that may be executed by the processor. In this case, the computer-readable recording medium may include a magnetic storage medium (for example, read-only memory (ROM), random-access memory (RAM), a floppy disk, a hard disk), an optical reading medium (for example, a compact disc read-only memory (CD-ROM), a digital versatile disc (DVD), and the like. The computer-readable recording medium may also be distributed over network-coupled computer systems so that the computer-readable code is stored and executed in a distributive manner. The computer-readable recording medium may be read by a computer, stored in a memory, and executed in the processor.

The embodiments may be described in terms of functional block components and various processing operations. Such functional blocks may be implemented as any number of hardware and/or software components configured to perform the specified functions. For example, the embodiments may employ various integrated circuit (IC) components, e.g., memory elements, processing elements, logic elements, look-up tables, and the like, which may carry out a variety of functions under the control of one or more microprocessors or other control devices. Similarly, where the elements are implemented using software programming or software elements, the present disclosure may be implemented with any programming or scripting language such as C, C++, Java, assembler language, or the like, with the various algorithms being implemented with any combination of data structures, objects, processes, routines or other programming elements. Functional aspects may be implemented in algorithms that are executed on one or more processors. Furthermore, the present disclosure may employ any number of techniques in the related art for electronics configuration, signal processing and/or control, data processing and the like. Terms such as “mechanism,” “element,” “means,” and “configuration” are used broadly and are not limited to mechanical or physical embodiments. The terms may include software routines in conjunction with processors, etc.

The particular implementations shown and described herein are illustrative examples of the present disclosure and are not intended to otherwise limit the scope of the present disclosure in any way. For the sake of brevity, conventional electronics, control systems, software development and other functional aspects of the systems may not be described in detail. Furthermore, connecting lines or connectors between various elements shown in the drawing are intended to illustratively represent functional relationships and/or physical or logical couplings between the various elements. It will be noted that many alternative or additional functional relationships, physical connections or logical connections may be present in a practical device.

Use of the terms “the” and similar references in the context of describing the present disclosure (especially in the context of the following claims) may be construed to cover both the singular and the plural. Furthermore, recitation of ranges of values herein are merely intended to serve as a shorthand method of referring individually to each separate value falling within the range, unless otherwise indicated herein, and each separate value is incorporated into the detailed description. The steps of all methods described herein may be performed in any suitable order unless otherwise indicated herein or otherwise clearly contradicted by context. The present disclosure is not limited to the described order of steps.

Example embodiments of the present disclosure have been shown and described. While the present disclosure has been particularly shown and described with reference to preferred embodiments thereof, it will be understood by those skilled in the art that various changes in form and details may be made therein without departing from the spirit and scope of the present disclosure as defined by the appended claims. The embodiments will be considered in descriptive sense only and not for purposes of limitation. Therefore, the scope of the present disclosure is defined not by the detailed description but by the appended claims, and all differences within the scope will be construed as being included in the present disclosure.

What is claimed is:

1. A method for predicting amino acid substitutions at a site of interest to generate an enzyme variant optimized for a biochemical reaction, performed in silico by at least one processor operably connected to a memory device, the method comprising:
receiving input information regarding a structure of an enzyme and a site of interest of the enzyme in proximity to a bound ligand;
identifying a functional atom of a wild type (WT) amino acid at the site of interest and a functional atom of the bound ligand;
confirming properties of the functional atom of the WT amino acid and the functional atom of the bound ligand;
detecting a presence or an absence of an interaction between the functional atom of the WT amino acid and the functional atom of the bound ligand;
selecting alternative amino acids according to a result of the detecting of the presence or the absence of the interaction;
determining a score for each of the selected alternative amino acids;
ranking the selected alternative amino acids, based on the scores; and
predicting, for optimizing the enzyme, substitutions of alternative amino acids having high rankings from among the selected alternative amino acids.

2. The method of claim 1,
wherein the selecting of the alternative amino acids according to the result of the detecting the presence or the absence of the interaction comprises,
when the presence of the interaction is detected:
identifying a type of the detected interaction;
selecting, from a knowledge library, alternative amino acids having interactions similar to the detected interaction; and
re-selecting the alternative amino acids from the selected alternative amino acids, based on a distance between the functional atom of the WT amino acid and the functional atom of the bound ligand and a size of the alternative amino acids; and
when the absence of the interaction is detected,
selecting the alternative amino acids, based on at least one of the distance between the functional atom of the WT amino acid and the functional atom of the bound ligand, a distance between a Cα atom of the WT amino acid and the functional atom of the ligand, and a nature of the functional atom of the bound ligand.

3. The method of claim 2,
wherein the selecting of the alternative amino acids having interactions similar to interactions identified from the knowledge library comprises:
selecting a set of amino acids based on a preference order assigned according to types of the identified interactions and selecting alternative amino acids from the selected set of amino acids.

4. The method of claim 3,
wherein the selecting of the alternative amino acids from the selected set of the amino acids comprises:
selecting similar-sized amino acids from the knowledge library when the distance between the functional atom of the WT amino acid and the functional atom of the bound ligand is within a predefined cutoff size range;
selecting smaller-sized amino acids from the knowledge library when the distance between the functional atom of the WT amino acid and the functional atom of the bound ligand is less than the predefined cutoff size range; and
selecting larger-sized amino acids from the knowledge library when the distance between the functional atom of the WT amino acid and the functional atom of the bound ligand is greater than the predefined cutoff size range.

5. The method of claim 2,
wherein the knowledge library comprises at least one of a structure of the amino acids, a list of the functional atoms, a binding pocket of the enzyme, amino acids and interaction types, amino acids and preferred secondary structures, amino acids and the number of hydrogen bonds, various physico-chemical properties, substitution probability in homologues, and an environment-specific substitution matrix.

6. The method of claim 1,
wherein the confirming of the properties of the functional atom of the WT amino acid and the functional atom of the bound ligand comprises:
confirming characteristics of the functional atom of the WT amino acid and the functional atom of the bound ligand, the distance between the functional atom of the WT amino acid and the functional atom of the bound ligand, and the distance between the Cα atom of the WT amino acid and the functional atom of the bound ligand.

7. The method of claim 6,
wherein the properties of the functional atom of the WT amino acid and the functional atom of the bound ligand is polar, non-polar, or aromatic.

8. The method of claim 1,
wherein the identifying of the functional atom of the WT amino acid comprises:
selecting at least one of a polar atom of the WT amino acid, a non-polar atom of the WT amino acid, centroids of polar atoms of a polar WT amino acid, centroids of non-polar atoms of a non-polar WT amino acid, a user-defined atom, and an atom based on a result of assessing of the input information for the knowledge library.

9. The method of claim 1,
wherein the identifying of the functional atom of the bound ligand comprises:
calculating a distance between the functional atom of the WT amino acid and at least one atom of the bound ligand; and
selecting, based on the calculated distance, at least one of an atom of the bound ligand in a shortest distance from the functional atom of the WT amino acid and an atom of the bound ligand in a predefined distance from the functional atom of the WT amino acid as the functional atom of the bound ligand.

10. The method of claim 1,
wherein the interaction between the functional atom of the WT amino acid and the functional atom of the bound ligand is one of an aromatic interaction, a polar interaction, a hydrophobic interaction, an electrostatic interaction, or a user-defined interaction.

11. The method of claim 1,
wherein, when the absence of the interaction detected,
a set of alternative amino acids having a property similar to the property of the functional atom of the ligand is selected.

12. The method of claim 11,
wherein a distance between the functional atom of the WT amino acid and the functional atom of the ligand is used for confirming an orientation of a side chain of the WT amino acid to the ligand to re-select the alternative amino acids having appropriate sizes when a distance between the Cα atom of the WT amino acid and the functional atom of the ligand is greater than the distance between the functional atom of the WT amino acid and the functional atom of the ligand.

13. The method of claim 11,
wherein a distance between the Cα atom of the WT amino acid and the functional atom of the ligand is used for identifying an orientation of a side chain of the WT amino acid apart from the ligand to re-select alternative amino acids having appropriate sizes when a distance between the Cα atom of the WT amino acid and the functional atom of the ligand is less than or equal to the distance between the functional atom of the WT amino acid and the functional atom of the ligand.

14. The method of claim 11,
wherein at least one of a distance between a Cα atom of the WT amino acid and the functional atom of the ligand or a distance between the functional atom of the WT amino acid and the functional atom of the ligand is used to re-select the alternative amino acids from the set of alternative amino acids.

15. The method of claim 1,
the determining of the scores for the selected alternative amino acids comprises calculating a weighted average of a volume, a polarity index, a total number of hydrogen bonds generated by the alternative amino acids, secondary structure propensity, substitution probability in homologues, environment-dependent substitution probability, substitution frequency in user-defined homologues, and physico-chemical properties of a user-defined amino acid.

16. The method of claim 1,
wherein, in the ranking of the selected alternative amino acids, based on the scores, an alternative amino acid having a lower score corresponds to a higher rank.

17. The method of claim 1, further comprising:
predicting an amino acid substitution for each site in a binding pocket of the enzyme; and
generating the enzyme variant optimized for the biochemical reaction, based on the amino acid substitution.

18. The method of claim 1, further comprising:
assessing compatibility for each site in a binding pocket of the enzyme for each ligands; and
prioritizing the ligands based on the assessed compatibility.

19. A device for in-silico predicting an amino acid substitution at a site of interest to generate an enzyme variant optimized for a biochemical reaction, the device comprising:
a memory storing instructions; and
a processor connected to the memory,
wherein the processor, upon execution of the instructions, performs:
receiving input information regarding the site of interest of an enzyme in proximity to a bound ligand and a structure of the enzyme;
identifying a functional atom of a wild type (WT) amino acid in the site of interest and a functional atom of the ligand;
confirming properties of the functional atom of the WT amino acid and the functional atom of the ligand;
detecting a presence or an absence of an interaction between the functional atom of the WT amino acid and the functional atom of the ligand;
selecting alternative amino acids according to a result of the detecting of the presence or the absence of the interaction;
determining a score for each of the selected alternative amino acids;
ranking the alternative amino acids, based on the scores; and
predicting, for optimizing the enzyme, substitutions of alternative amino acids having high rankings from among the selected alternative amino acids.

20. The device of claim 19,
wherein the selecting of the alternative amino acids according to a result of the detecting of the presence or the absence of the interaction further comprises,
when the presence of the interaction is detected:
confirming types of the detected interactions;
selecting, from a knowledge library, alternative amino acids having interactions similar to a type of the detected interaction; and
re-selecting the alternative amino acids, from among the selected alternative amino acids, based on a distance between the functional atom of the WT amino acid and the functional atom of the ligand and a size of the alternative amino acids, and
when the absence of the interaction is detected:
selecting the alternative amino acids, based on at least one of the distance between the functional atom of the WT amino acid and the functional atom of the ligand, a distance between the Cα atom of the WT amino acid and the functional atom of the ligand, and properties of the functional atom of the ligand.

21. The device of claim 19,
wherein the processor further performs:
prediction of amino acid substitutions for sites in a binding pocket of the enzyme; and
generation of an enzyme variant optimized for a biochemical reaction, based on the amino acid substitutions.

22. The device of claim 19,
wherein the processor further performs:
assessment of compatibility with respect to each ligand of sites in a binding pocket of the enzyme; and
prioritization of the ligands, based on the assessment of compatibility.
